(19) United States
(12) Patent Application Publication
(10) Pub. No.: US 2026/0038641 A1
(43) Pub. Date: Feb. 5, 2026

(54) METHOD, APPARATUS, DEVICE, AND READABLE STORAGE MEDIUM FOR DETERMINING A MOLECULAR FORCE FIELD PARAMETER

(71) Applicants: DOUYIN VISION CO., LTD., Beijing (CN); Lemon Inc., Grand Cayman (KY)

(72) Inventors: Tianze Zheng, Beijing (CN); Wen Yan, Los Angeles, CA (US); Chi Xu, Beijing (CN); Xu Han, Beijing (CN)

(21) Appl. No.: 19/288,126

(22) Filed: Aug. 1, 2025

(30) Foreign Application Priority Data
Aug. 2, 2024 (CN) 202411060355.X

(51) Int. Cl. G16C 10/00

(52) U.S. Cl. CPC G16C 10/00

(57) ABSTRACT

Provided in the disclosure a method, an apparatus, a device, and a readable storage medium for determining a molecular force field parameter. A method includes acquiring structural information of a target molecule; encoding the structural information to obtain an encoded representation of the target molecule, where the encoded representation includes encoded representations corresponding to a plurality of hierarchical levels, a designated hierarchical level in the plurality of hierarchical levels indicates feature information of at least one atom included in the target molecule, and the feature information indicated by the designated hierarchical level includes additional feature information based on feature information indicated by an immediately preceding hierarchical level; and determining, based on the encoded representation of the target molecule, a molecular force field parameter corresponding to the target molecule.

### CROSS-REFERENCE

This application claims the benefit of Chinese Patent Application No. 202411060355.X, filed Aug. 2, 2024, entitled “Method, Apparatus, Device, and Readable Storage Medium for Determining a Molecular Force Field Parameter”, the entirety of which is incorporated herein by reference.

### TECHNICAL FIELD

Example embodiments in the disclosure generally relate to the field of computing, and in particular, to a method, an apparatus, a device, and a readable storage medium for determining a molecular force field parameter.

### BACKGROUND

In the field of chemistry and bioinformatics, representation and analysis of molecular structures are important steps in studying molecular properties and behaviors. Molecular force field is a tool for describing the potential energy surface of a molecular, that is, the three-dimensional coordinates of each atom in a given molecule, which may predict the energy of the molecule. Common molecular force field generation methods manually formulate rules, specify atomic types according to chemical environments, and obtain force field parameters such as bonds, bond angles and dihedral angles according to atom type combinations. However, these methods have problems such as limited atomic types, inflexible parameter binding, and incorrect identification.

### SUMMARY

In a first aspect in the disclosure, a method for determining a molecular force field parameter is provided. The method may include: acquiring structural information of a target molecule; encoding the structural information to obtain an encoded representation of the target molecule, where the encoded representation includes encoded representations corresponding to a plurality of hierarchical levels, a designated hierarchical level in the plurality of hierarchical levels indicates feature information of at least one atom included in the target molecule, and the feature information indicated by the designated hierarchical level includes additional feature information based on feature information indicated by an immediately preceding hierarchical level; and determining, based on the encoded representation of the target molecule, a molecular force field parameter corresponding to the target molecule.

In a second aspect in the disclosure, an apparatus for determining a molecular force field parameter is provided. The apparatus may include: a structural information determining module configured to acquire structural information of a target molecule; an encoding module configured to encode the structural information to obtain an encoded representation of the target molecule, where the encoded representation includes encoded representations corresponding to a plurality of hierarchical levels, a designated hierarchical level in the plurality of hierarchical levels indicates feature information of at least one atom included in the target molecule, and the feature information indicated by the designated hierarchical level includes additional feature information based on feature information indicated by an immediately preceding hierarchical level; and a molecular force field parameter determining module configured to determine, based on the encoded representation of the target molecule, a molecular force field parameter corresponding to the target molecule.

In a third aspect in the disclosure, an electronic device is provided. The device includes at least one processor; and at least one memory, where the at least one memory is coupled to the at least one processor and stores instructions for execution by the at least one processor, and the instructions, when executed by the at least one processor, cause the electronic device to perform the method according to the first aspect.

In a fourth aspect in the disclosure, a computer-readable storage medium is provided. A computer program is stored on the medium, and the computer program is executable by a processor to implement the method according to the first aspect.

In a fifth aspect in the disclosure, a computer program product is provided. The computer program product includes computer-executable instructions, where the computer-executable instructions, when executed by a processor, implement the method according to the first aspect.

It should be understood that the content described in this section is not intended to limit the key features or important features of the embodiments in the disclosure, nor is it intended to limit the scope of the disclosure. Other features in the disclosure will become easy to understand through the following description.

### BRIEF DESCRIPTION OF THE DRAWINGS

The above and other features, advantages and aspects of embodiments in the disclosure become more apparent with reference to the following detailed description when taken in conjunction with the drawings. In the drawings, the same or similar reference numerals refer to the same or similar elements, where:

FIG. 1 illustrates a schematic diagram of an example environment in which embodiments in the disclosure may be implemented;

FIG. 2 illustrates a flowchart of a method for determining a molecular force field parameter according to some embodiments in the disclosure;

FIG. 3 illustrates an example diagram of a target molecule according to some embodiments in the disclosure;

FIG. 4 illustrates an example diagram of an example structural fragment according to some embodiments in the disclosure;

FIG. 5 illustrates a schematic diagram of an example structural fragment according to some other embodiments in the disclosure;

FIG. 6 illustrates a schematic diagram of an example structural fragment according to some other embodiments in the disclosure;

FIG. 7 illustrates a schematic structural block diagram of an apparatus for determining a molecular force field parameter according to some embodiments in the disclosure; and

FIG. 8 illustrates a block diagram of an electronic device that may implement one or more embodiments in the disclosure.

### DETAILED DESCRIPTION OF EMBODIMENTS

The embodiments in the disclosure will be described in more detail below with reference to the drawings. Although some embodiments in the disclosure are shown in the drawings, it should be understood that the disclosure may be implemented in various forms and should not be interpreted as limited to the embodiments set forth herein, and on the contrary, these embodiments are provided for a more thorough and complete understanding of the disclosure. It should be understood that the drawings and the embodiments in the disclosure are only for illustrative purposes, and are not intended to limit the scope of protection of the disclosure.

In the description of the embodiments in the disclosure, the term “include/comprise” and similar terms should be understood as open-ended inclusions, that is, “include/comprise but not limited to”. The term “based on” should be understood as “based at least in part on”. The term “one embodiment” or “the embodiment” should be understood as “at least one embodiment”. The term “some embodiments” should be understood as “at least some embodiments”. Other explicit and implicit definitions may also be included below.

In this article, unless explicitly stated, performing a step “in response to A” does not mean that the step is performed immediately after “A”, but may include one or more intermediate steps.

It should be understood that the data involved in the technical solution in the disclosure (including but not limited to the data itself, the acquisition, use, storage or deletion of the data) should comply with the requirements of corresponding laws and regulations and relevant regulations.

It should be understood that before using the technical solutions disclosed in the embodiments in the disclosure, relevant users should be informed of the type, use scope, use scenario, etc. of the information involved in the disclosure and authorization of the relevant users should be obtained through appropriate methods according to relevant laws and regulations, where the relevant users may include any type of rights subject, such as individuals, enterprises, and groups.

For example, in response to receiving an active request from a user, prompt information is sent to the relevant user to explicitly prompt the relevant user that the operation requested to be performed will need to obtain and use the information of the relevant user, so that the relevant user may independently select whether to provide information to software or hardware such as an electronic device, an application, a server or a storage medium that performs the operation of the technical solution in the disclosure according to the prompt information.

As an optional but non-restrictive embodiment, a manner of sending prompt information to the relevant user in response to receiving an active request from the relevant user may be, for example, a pop-up window, and the prompt information may be presented in the pop-up window in the form of text. In addition, the pop-up window may also carry a selection control for the user to select “agree” or “disagree” to provide information to the electronic device.

It should be understood that the above-mentioned process of notifying and obtaining user authorization is only illustrative and does not constitute a limitation on the embodiment in the disclosure, and other manners that meet relevant laws and regulations may also be applied to the embodiment in the disclosure.

As used herein, the term “model” may learn an association between a corresponding input and output from training data, so that after the training is completed, a corresponding output may be generated for a given input. The generation of the model may be based on a machine learning technique. Deep learning is a machine learning algorithm that processes an input and provides a corresponding output by using a plurality of processors. A neural network model is an example of a deep learning-based model. Herein, the “model” may also be referred to as a “machine learning model”, a “learning model”, a “machine learning network” or a “learning network”, which terms are used interchangeably herein.

FIG. 1 shows a schematic diagram of an example environment 100 in which embodiments in the disclosure may be implemented. The environment 100 includes an electronic device 110. It is expected to use such an electronic device 110 to determine a molecular force field parameter 120 corresponding to a target molecule 102. To this end, in some embodiments, a machine learning model 150 may be deployed in the electronic device 110 for determining the molecular force field parameter 120 of the target molecule.

As an example, the electronic device 110 may include any computing system with computing power, such as various computing devices/systems, terminal devices, server-side devices, etc. The terminal device may be any type of mobile terminal, fixed terminal or portable terminal, including a mobile phone, a desktop computer, a laptop computer, a notebook computer, a netbook computer, a tablet computer, a media computer, a multimedia tablet, a palmtop computer, a portable game terminal, a game device, or any combination of the foregoing, including accessories and peripherals of these devices, or any combination thereof. The server-side device may be an independent physical server, or a server cluster or distributed system composed of a plurality of physical servers, or may also be a cloud server that provides basic cloud computing services such as cloud services, cloud databases, cloud computing, cloud functions, cloud storage, network services, cloud communication, middleware services, domain name services, security services, content delivery networks, and big data and artificial intelligence platforms. The server-side device may, for example, include a computing system/server, such as a mainframe, an edge computing node, a computing device in a cloud environment, and the like. It should be understood that the structure and function of each element in the environment 100 are described for illustrative purposes only, and are not intended to imply any limitation on the scope of the disclosure.

At present, in the field of molecular force field parameter determination, a commonly used method is to specify an atomic type for each atom in a target molecule according to its chemical environment based on a manually formulated rule system, and obtain corresponding molecular force field parameters such as bonds, bond angles and dihedral angles according to atom type combinations. These molecular force field parameters are used to calculate the energy of the molecule in a specific three-dimensional structure, for example, a C—H bond may indicate a bond length between a carbon atom and a hydrogen atom. The molecular force field parameter is used to describe the length of the C—H bond and its influence on the energy. Similarly, a bond angle or a dihedral angle may describe atoms forming the bond angle or the dihedral angle, a size of the bond angle or the dihedral angle, and an influence of the bond angle or the dihedral angle on the energy. The molecular force field parameter may describe an energy change of the molecular fragment, thereby predicting the stability and reactivity of the molecule. In traditional force field models, the physicochemical properties of the molecule may be more accurately simulated by assigning different parameters to these specific structures (such as bonds, bond angles and dihedral angles).

There are three major problems in the commonly used method. One problem is that atomic types corresponding to the manually formulated rule system are very limited. As dataset grows, it is very difficult for the number of force field parameters to grow at a synchronous speed. For example, for common atomic types such as carbon, hydrogen, oxygen, and nitrogen, corresponding molecular force field parameters may be formulated. However, if a more complex molecular structure or a new molecular result is encountered, it may not be possible to handle it.

The second problem is that molecular force field parameters such as bonds and bond angles are strongly bound to atomic types, which lack flexibility. For example, for ethanol (CH3CH2OH), there will be corresponding molecular force field parameters to describe the carbon-oxygen bond in the ethanol. However, if a molecule similar to ethanol such as methyl ether (CH3OCH3) is encountered, the same molecular force field will no longer apply.

The third problem is that the bond type obtained by combining the atomic types may cause incorrect identification of certain specific chemical structures, thereby affecting the accuracy. For example, the carbon-carbon bond in the benzene ring has a conjugated feature, rather than a simple single bond or double bond. In the commonly used method, this special structure may not be correctly recognized, resulting in false force field parameter matching.

For example, any molecule may be represented in a graph, that is, G=(V,E), where V may represent a set of atoms in the molecule, and E may represent a set of chemical bonds between two atoms. vi may represent an ith atom in the molecule, and the atom has features such as element type, number of connections, aromaticity, the minimum ring, number of ring connections, etc., and vi∈V.E may represent a chemical bond between an ith atom vi and a jth atom vj, and the chemical bond has features such as bond order (single, double, triple or aromatic bond) and whether it is in a ring. A local part of the molecule may be represented as a representation of an induced subgraph, that is, G′=G[V′]=(V′,E′), ∇ vi′EV′, ∇ eij′∈E′. G′ may represent an induced subgraph, that is, a local region or substructure of the molecule. V′ may represent a set of nodes in the induced subgraph G′, that is, atoms in the local region. E′ may represent a set of edges in the induced subgraph G′, that is, chemical bonds in the local region. vi′ may be an ith atom in the induced subgraph G′, and has corresponding partial features. eij′ may be a chemical bond between an atom vi′ and an atom vj′ in the induced subgraph G′, and has corresponding partial features.

Only the representation of fully connected subgraphs is considered. In a chemical environment, this means that all atoms and the bonds between them are interconnected, and there are no isolated atoms or disconnected bonds. Based on this, the chemical environment may be defined as: f(G′)=(fv(V′),fe(E′)), where fv and fe act on atoms and bonds respectively, retaining their partial features. fv(V′) may form a feature set corresponding to the partial features of all atoms in the subgraph, and fe(E′) may form a feature set corresponding to the partial features of all chemical bonds in the subgraph.

f(G′) may be understood as a set of different subgraphs. Considering that G′ must satisfy the topological relationship required by f(G′) and the features of atoms and bonds, G′ Ef(G′). At the same time, since fv and fe only retain partial features of atoms and bonds, if any subgraph G″ is different from G′ only in the unreserved features, then G″ Ef(G′).

The essence of the force field is to establish a mapping between f(G′) and the force field parameter θ. However, if f(G′) itself is directly recorded, for any given molecule, graph matching needs to be performed on all the recorded chemical environments. The graph matching itself is a complex problem, and it also needs to be multiplied by the number of recorded force field parameters. It can be seen that this process is very inefficient.

With the development of computing power, the number of molecules obtained by quantitative calculation may reach the magnitude of millions, and the number of conformations reaches the magnitude of tens of millions. Correspondingly, the force field also needs more parameter representations to provide accurate modeling for these different chemical environments. Therefore, there are two main problems: how to automatically generate and record the description of f(G′) and the corresponding force field parameters based on massive data; and how to efficiently query the corresponding parameters for a given chemical structure when the number of force field parameters are extremely large.

Embodiments in the disclosure propose a solution for determining a molecular force field parameter. According to the solution, structural information of a target molecule is acquired; encoding is performed on the structural information to obtain an encoded representation of the target molecule, where the encoded representation includes encoded representations corresponding to a plurality of hierarchical levels, a designated hierarchical level in the plurality of hierarchical levels indicates feature information of at least one atom included in the target molecule, and the feature information indicated by the designated hierarchical level includes additional feature information based on feature information indicated by an immediately preceding hierarchical level; and a molecular force field parameter corresponding to the target molecule is determined based on the encoded representation of the target molecule.

In this way, atoms and their chemical environments can be flexibly described through the encoded representations of the plurality of hierarchical levels, so that the problem of limited atomic types is solved. The feature information indicated by each hierarchical level is additional feature information based on the feature information indicated by the immediately preceding hierarchical level, which makes parameter generation and matching more precise and flexible. In addition, through the encoded representations corresponding to the plurality of hierarchical levels, the complexity of the molecular force field parameter matching is reduced, the efficiency is improved, and the matching process of the molecular force field parameter is optimized.

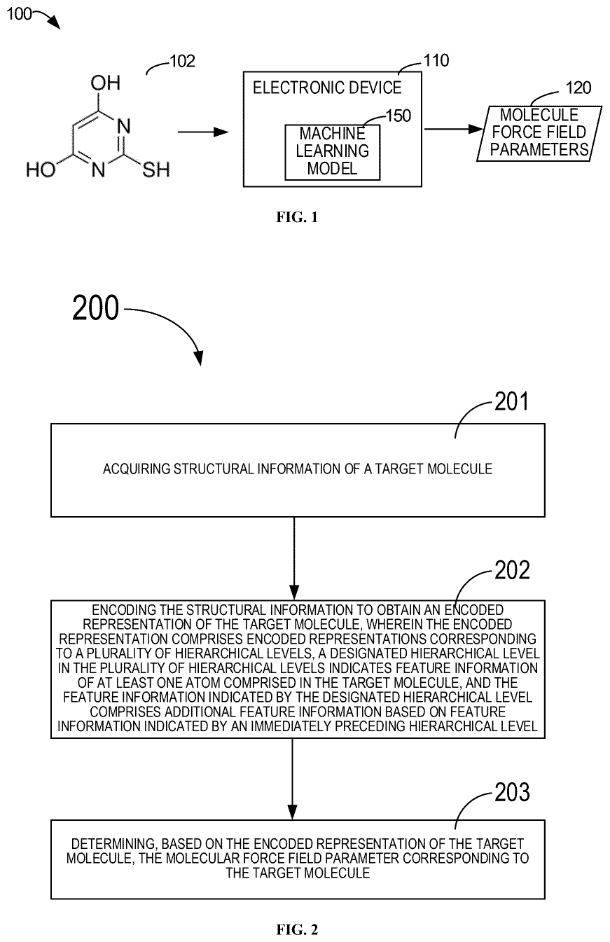

The embodiments in the disclosure will be described in detail below with reference to the drawings. FIG. 2 shows a flowchart of a molecular encoding process 200 according to some embodiments in the disclosure. The process 200 may be implemented at the electronic device 110. For ease of discussion, the process 200 will be described below with reference to FIG. 1.

As shown in FIG. 2, at block 201, the electronic device 110 may acquire structural information of a target molecule by traversing atoms in the target molecule. For example, a root node may be specified in the target molecule, so that the atoms in the target molecule are traversed based on the root node.

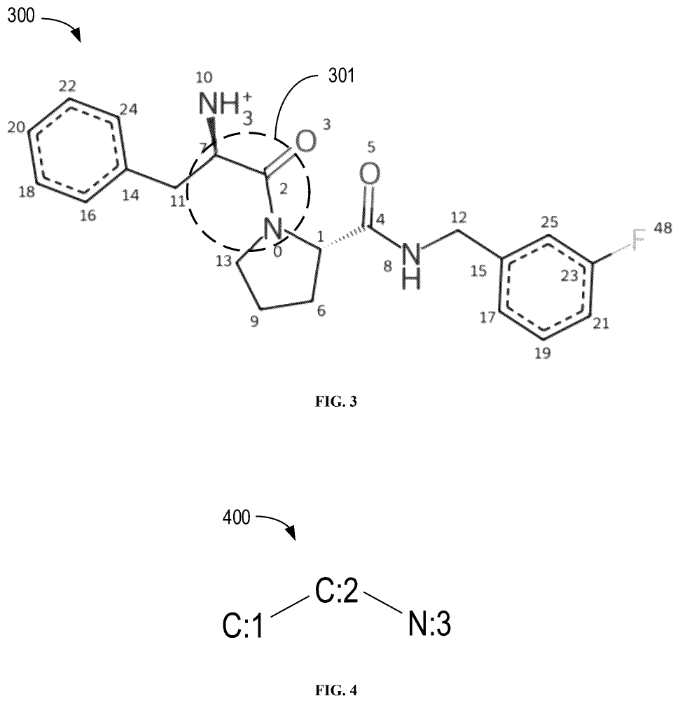

FIG. 3 shows a schematic diagram of a target molecule 300 according to some embodiments in the disclosure. Taking traversing one molecular structural fragment of the target molecule 300 as an example, a node labeled 2 may be used as the root node. The root node includes 3 branches, branch 1 is traversed to a node labeled 7, branch 2 is traversed to a node labeled 3, and branch 3 is traversed to a node labeled 0. That is, each atom included in the molecular structural fragment may be traversed from the root node, and structural information may be generated. The structural information may include attribute information of the atoms traversed along the traversal path. As an example, the attribute information may indicate feature information of the atom, for example, the feature information may indicate an element type, aromaticity, a number of connections, a number of ring connections, the smallest ring it is in, etc. of the atom. For example, for the atom labeled 3, the attribute information of the atom may indicate that the atom is labeled 3, the element type of the atom is an oxygen atom, and the atom is bonded to the atom labeled 2 by a double bond. It should be noted that the presentation of the molecular fragment structure involved in the disclosure and the hydrogen atom are omitted in the encoding process, and the purpose of the omission is to save the encoding amount of the encoding.

At block 202, the electronic device 110 encodes the structural information to obtain an encoded representation of the target molecule, where the encoded representation includes encoded representations corresponding to a plurality of hierarchical levels, a designated hierarchical level in the plurality of hierarchical levels indicates feature information of at least one atom included in the target molecule, and the feature information indicated by the designated hierarchical level includes additional feature information based on feature information indicated by an immediately preceding hierarchical level.

Based on the structural information of the target molecule, encoding may be performed on the structural information to obtain the encoded representation. The encoded representation may include encoded representations of the plurality of hierarchical levels. Each hierarchical level may include encoding at least one piece of feature information of the target molecule, and the feature information indicated by a current hierarchical level includes additional feature information based on feature information indicated by an immediately preceding hierarchical level.

For example, if the feature information indicated by the immediately preceding hierarchical level of the current hierarchical level includes feature information of an atom A, the feature information of the current hierarchical level may include feature information of the atom A and an atom B, where the atom B is bonded to the atom A. For another example, if the feature information indicated by the immediately preceding hierarchical level of the current hierarchical level includes feature information of an element type of an atom A, the feature information of the current hierarchical level may include feature information of the element type and the number of connections of the atom A. The number of hierarchical levels may be set according to actual situations, and in some embodiments, the number of hierarchical levels may be set to 3.

The encoding may be performed on the structural information of the target molecule using an encoding rule corresponding to SMiles Arbitrary Target Specification (SMARTS) to obtain a string that describes a local chemical language, and the string may be used by the force field for parameter matching. In the disclosure, some functions of SMARTS may be retained. Specifically, for the description of the atom, only the features that may be described by one number or character, such as the element type (corresponding to the string “#”), the number of connections (corresponding to the string “X”), the aromaticity (corresponding to the string “A/a”), the smallest ring (corresponding to the string “r”), and the number of ring connections (corresponding to the string “x”) are retained. For the description of the chemical bonds, features such as bond order (for example, symbols “−”, “=”, “#” and “:” represent single bond, double bond, triple bond and aromatic bond, respectively) and whether it is in a ring (symbols “@” and “!@” may represent bonds in the ring and bonds outside the ring, respectively) may be retained, and any bond may also be represented by symbol “−”. In addition, fuzzy features are deleted, for example, if a feature is described by a numerical range, it may be considered as a fuzzy feature and thus may be discarded. As an example, for the ring, if the encoding rule includes less than or greater than an N-membered ring (N is a positive integer), it belongs to a feature described by a numerical range, which may be discarded. On the contrary, if it is a definite value such as a three-membered ring or a five-membered ring, it may be retained.

Based on the above processes, encoded representations (SMARTS) of different hierarchical levels may be constructed. If more descriptions of features such as atoms or chemical bonds are added to the atoms based on the original encoded representation, or atoms and bonds are added, a new encoded representation may be obtained. For example, the encoded representation corresponding to the atom indicated by the top hierarchical level is “[#6]-[#6]”. In the second hierarchical level, the encoded representation of the second hierarchical level may be obtained by adding a feature of the number of connections of the atom to the atom of the top hierarchical level. For example, the encoded representation of the second hierarchical level may be “[#6X4]-[#6X4]”. The structure that may be matched by the encoded representation corresponding to the new hierarchical level is necessarily a subset of the encoded representation that may be matched by the encoded representation corresponding to the immediately preceding hierarchical level. Secondly, it may be determined whether the encoded representations of two molecules are the same. The encoded representations of the two molecules are restored to graph structures, and if the two graphs are isomorphic and the corresponding descriptors of the atoms and bonds are the same, the encoded representations of the two molecules are considered to be the same.

At block 203, the electronic device 110 determines, based on the encoded representation of the target molecule, the molecular force field parameter corresponding to the target molecule. The encoded representation of the target molecule may be used as a language to describe a molecular structure. The encoded representation may represent the feature information of the target molecule. In some embodiments, the encoded representation describes the complex target molecule through a specific string pattern.

The force field parameter may be used to describe the interaction between atoms in the molecule, including bond length, bond angle, dihedral angle, non-bond interaction, and the like. These force field parameters are determined by experimental data and computational chemistry methods, and are used to simulate the molecular structure and behavior.

A matching relationship between the force field parameter and the encoded representation may be pre-constructed. The encoded representation and its corresponding force field parameter are recorded in the matching relationship. By using the encoded representation to reflect the structure in the molecule, the electronic device 110 may find the force field parameter corresponding to the encoded representation according to the matching relationship table, and apply it to the molecular simulation.

The encoded representation of the target molecule includes encoded representations corresponding to the plurality of hierarchical levels. Therefore, in the process of determining the molecular force field parameter, it may be determined hierarchically. For example, firstly, from the top hierarchical level, the encoded representation of the top hierarchical level is matched with the molecular force field parameter. Secondly, the matching of the second hierarchical level, the matching of the third hierarchical level, etc. are performed, until the corresponding sub-force field parameter cannot be matched. Finally, the force field parameter successfully matched at the last time may be used as the molecular force field parameter corresponding to the target molecule.

Through the above process, the structural information of the target molecule is encoded hierarchically, which may refine the chemical environment of the target molecule, thereby capturing the features of the target molecule more accurately. This hierarchical encoding can ensure that all relevant chemical features of the target molecule are included, thereby solving the problems of limited atomic types and inflexible binding of molecular force field parameters. By performing the matching of the force field parameter through hierarchical encoding, the corresponding molecular force field parameter can be efficiently found. For example, in the traditional matching manner, the complexity corresponding to the matching process is linearly related to the input scale. That is, if the input scale is doubled, the complexity corresponding to the matching process will also double. That is, in the traditional matching manner, the complexity corresponding to the matching process may be expressed as O(n), where n may correspond to the input scale. However, if the manner of determining the molecular force field parameter hierarchically corresponding to the disclosure is adopted, the time consumed for matching is proportional to the input scale. That is, if the input scale is doubled, the time consumed for matching only increases by a constant unit. That is, in the manner corresponding to the disclosure, the complexity corresponding to the matching process may be expressed as O(log(n)), where n may correspond to the input scale. In this way, the process of determining the molecular force field parameter is simplified.

In addition, through the above process, the advantage may be better exerted in the scenario where the number of molecules is in the order of millions or even tens of millions. For example, for millions of molecules, if a one-by-one matching method is adopted, the amount off calculation will be very large and it will be time-consuming. The matching method of a plurality of hierarchical levels can significantly reduce the number of matching and the complexity of calculation through hierarchical matching. Specifically, the structure of a plurality of hierarchical levels may decompose the complex matching process into a plurality of relatively simple hierarchical matching, and the matching of each hierarchical level only requires processing of a small amount of data, which can greatly improve the matching efficiency. At the top hierarchical level, the rough structural information is matched, while at the deeper hierarchical level, the more detailed and specific structural information is matched. This hierarchical refinement matching manner can ensure that the final matching result is more accurate and precise, which is particularly important for large-scale molecular data.

In an embodiment, the electronic device 110 encoding the structural information includes two cases. One case is to encode the top hierarchical level of the plurality of hierarchical levels, and the other is to encode other hierarchical levels except the top hierarchical level. For the top hierarchical level of the plurality of hierarchical levels, a first structural fragment in the target molecule is acquired, where the first structural fragment includes at least one atom. Encoding is performed on feature information of the at least one atom included in the first structural fragment to obtain an encoded representation corresponding to the top hierarchical level. For a non-top hierarchical level of the plurality of hierarchical levels, a second structural fragment in the target molecule is acquired, where the second structural fragment is determined based on a structural fragment corresponding to an immediately preceding hierarchical level of the non-top hierarchical level. Encoding is performed on feature information of an atom included in the second structural fragment to obtain an encoded representation corresponding to the non-top hierarchical level.

The feature information of different hierarchical levels indicates the atomic situation of the target molecule. For example, for the top hierarchical level of the plurality of hierarchical levels, the first structural fragment in the target molecule may be selected. The first structural fragment is usually a single atom or a simple combination of atoms. The selection of the first structural fragment in the target molecule may be selected according to a scenario, or may be selected according to a specified selection order.

Referring to FIG. 3, the first structural fragment 301 may be a structural fragment of the target molecule 300. FIG. 4 shows a schematic diagram of a topology 400 of the first structural fragment 301 according to some embodiments in the disclosure, and the first structural fragment 301 may be a basic structural fragment of the target molecule 300. In the example shown in FIG. 4, the first structural fragment 301 includes one carbon atom, and one nitrogen atom and another carbon atom connected to the carbon atom. Encoding is performed on the feature information of each atom in the first structural fragment to obtain the encoded representation corresponding to the top hierarchical level. As an example, the encoded representation corresponding to the top hierarchical level may include: “[#6X3x0:2](−[#6X4x0:1])-[#7X3x2:3]”. Correspondingly, the encoding may indicate the carbon atom (C:2) at the 2nd position, which is trivalent and not in a ring. The carbon atom (C:1) at the 1st position, which is tetravalent and not in a ring, is connected to the carbon atom (C:2) at the 2nd position. The nitrogen atom at the 3rd position, which is trivalent and has two lone pairs of electrons, is connected to the carbon atom (C:2) at the 2nd position.

The feature information of each atom in the first structural fragment may include a chemical element type representation, bonding condition, aromaticity, number of ring connections, etc. of the atom. These pieces of information will be used as the encoded representation of the top hierarchical level to describe the basic features of the target molecule.

For a non-top hierarchical level of the plurality of hierarchical levels, encoding may be performed based on the immediately preceding hierarchical level of the non-top hierarchical level. Specifically, the second structural fragment is acquired from the structural fragment corresponding to the previous hierarchical level, and a new atom or a combination of atoms is added based on the second structural fragment. Encoding is performed on the feature information of the extended atom to obtain the encoded representation corresponding to the non-top hierarchical level. Taking the non-top hierarchical level being the second hierarchical level after the top hierarchical level as an example, FIG. 5 shows a schematic diagram of a topology 500 of the second structural fragment according to some embodiments in the disclosure. The second structural fragment is a new structural fragment obtained by adding an atom based on the first structural fragment. In the example shown in FIG. 5, a new atom is added based on the first structural fragment 301, so that the second structural fragment is obtained. For example, the second structural fragment is added with a nitrogen atom bonded to the first carbon atom (C:1), and an oxygen atom bonded to the second carbon atom (C:2) to form a double bond. The encoding is performed on the second structural fragment to obtain the encoded representation corresponding to the second hierarchical level. As an example, the encoded representation corresponding to the second hierarchical level may be: “[#6X3x0:2](−[#6X4x0:1](−[#6X4x0])-[#7X4x0])(−[#7X3x2:3](−[#6X4x2])-[#6X4x2])=[#8X1x0]”.

Correspondingly, the encoding may indicate the carbon atom (C:2) at the 2nd position, which is trivalent and not in a ring. The carbon atom (C:1) at the 1st position, which is tetravalent and not in a ring, is connected to the carbon atom (C:2) at the 2nd position. The tetravalent carbon atom (C:4) connected to the first carbon atom (C:1) is not in a ring. The tetravalent carbon atom (C:4) is connected to a tetravalent nitrogen atom (N:5), which is not in a ring. The trivalent nitrogen atom at the 3rd position has two lone pairs of electrons. The third nitrogen atom is connected to a tetravalent carbon atom (C:6), and the tetravalent carbon atom (C:6) is further connected to a tetravalent carbon atom (C:7). The carbon atom (C:2) at the 2nd position also forms a double bond with a monovalent oxygen atom.

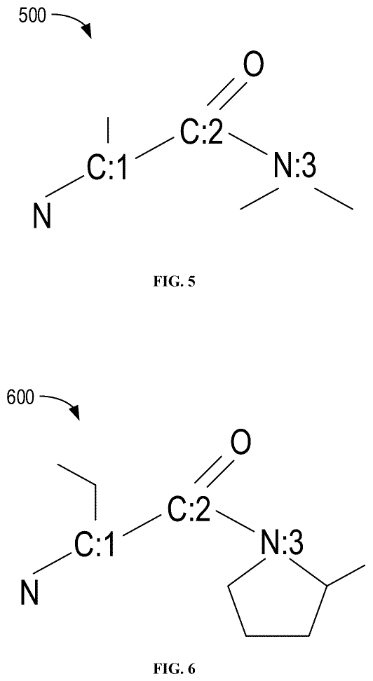

Based on the second hierarchical level, a third hierarchical level may also be included. The third hierarchical level may be a hierarchical level after the second hierarchical level. FIG. 6 shows a schematic diagram of a topology 600 of the third structural fragment according to some embodiments in the disclosure. In the example shown in FIG. 6, a new atom is added based on the second structural fragment, so that the third structural fragment is obtained. The third structural fragment is a new structural fragment obtained by adding an atom based on the second structural fragment. The encoding is performed on the third structural fragment to obtain the encoded representation corresponding to the third hierarchical level. As an example, the encoded representation corresponding to the third hierarchical level may be:

$$“ [ \#6 X 3 x 0 : 2 ] ( - [ \#6 X 4 x 0 : 1 ] ( - [ \#6 X 4 x 0 ] - [ \#6 X 3 x 2 ] ) - [ \#7 X 4 x 0 ] ) ( - [ \#7 X 3 x 2 : 3 ] ( - [ \#6 X 4 x 2 ] ( - [ \#6 X 3 x 0 ] ) - [ \#6 X 4 x 2 ] - 1 ) - [ \#6 X 4 x 2 ] - [ \#6 X 4 x 2 ] - 1 ) = [ \#8 X 1 x 0 ] ” .$$

The features indicated by the encoding are features further added based on the features indicated by the second hierarchical level, and the features are only for an example illustration, without posing any limitation on the actual situation, and thus will not be described in detail here.

Through the above encoding method for the molecular structural information, the encoded representations corresponding to the respective hierarchical levels may be obtained, and these encoded representations may not only accurately describe the basic features of the target molecule, but also expand layer by layer, comprehensively covering the complex chemical environment of the molecule. The encoded representation of each hierarchical level includes not only the feature information of the previous hierarchical level, but also additional new feature information, which makes the encoding result more detailed and accurate.

In an embodiment, the electronic device 110 encoding the structural information may further include: for a top hierarchical level of the plurality of hierarchical levels, acquiring a chemical element type representation of a given atom in at least one atom indicated by the top hierarchical level, where the chemical element type representation is a part of the feature information; and encoding the chemical element type representation of the given atom to obtain an encoded representation corresponding to the top hierarchical level. For a non-top hierarchical level of the plurality of hierarchical levels, acquiring a structural type representation of a given atom in at least one atom indicated by the top hierarchical level, where the structural type representation is a part of the feature information; and encoding the structural type representation and the chemical element type representation of the given atom to obtain an encoded representation corresponding to the non-top hierarchical level.

In addition to expanding the structural fragment of the target molecule between different hierarchical levels, the structural type of the target molecule may also be expanded between different hierarchical levels. For example, for a target molecule with relatively simple atomic composition, the atoms indicated by different hierarchical levels are the same, and the difference lies in the structural type representation of the atoms. For example, the atoms indicated by different hierarchical levels are the same, and all correspond to one carbon atom, and one nitrogen atom and another carbon atom connected to the carbon atom.

For the top hierarchical level of the plurality of hierarchical levels, a chemical element type (e.g., carbon, hydrogen, oxygen, etc.) of the atom in the target molecule is acquired. The chemical element type representation is encoded to obtain the encoded representation corresponding to the top hierarchical level. The encoded representation may describe the basic component of the molecule. As an example, the encoded representation corresponding to the top hierarchical level may include:

$$“ [ \#6 : 2 ] ( - [ \#6 : 1 ] ) - [ \#7 : 3 ] ” .$$

Correspondingly, the encoding may indicate the carbon atom (C:2) at the 2nd position. The carbon atom (C:1) at the 1st position is connected to the carbon atom (C:2) at the 2nd position. The nitrogen atom (N:3) at the 3rd position is connected to the carbon atom (C:2) at the 2nd position.

The reason why the encoded representation corresponding to the current embodiment is different from the encoded representation corresponding to the top hierarchical level in the foregoing example lies in the difference in the degree of detail. For example, the encoded representation corresponding to the top hierarchical level in the foregoing example includes features such as valence, ring information, atomic number, and lone pair electron information, while the encoded representation corresponding to the top hierarchical level in the current embodiment only includes features such as atomic number and atomic type. In this way, different scenarios may be handled, for example, including more feature representations may be applied to scenarios that require precise description and analysis. Including fewer features may be applied to scenarios that require rapid screening and preliminary matching.

For a non-top hierarchical level in the plurality of hierarchical levels, encoding may be performed based on the immediately preceding hierarchical level of the non-top hierarchical level. Taking the second hierarchical level as an example, at least one atom is selected based on the chemical element type representation of each atom in the previous hierarchical level (top hierarchical level), and the structural type representation of the atom is acquired. The structural type representation may include bond order feature information (for example, single bond, double bond, etc.), valence feature information (for example, divalent, trivalent, etc.), etc. The encoded representation corresponding to the second hierarchical level may be obtained by encoding the chemical element type representation and the bond order feature information of the atom. Taking the addition of the valence feature information as an example, the encoded representation corresponding to the second hierarchical level may include: “[#6X3:2](−[#6X4:1])-[#7X3:3]”.

Taking the third hierarchical level as an example, based on the chemical element type representation and the bond order feature information of the atom, additional feature information such as the number of ring connections and lone pair electron information may be added. The encoded representation corresponding to the third hierarchical level may be obtained by encoding the chemical element type representation, the bond order feature information, and the number of ring connections feature information. Taking the addition of features such as the number of ring connections and lone pair electron information as an example, the encoded representation corresponding to the third hierarchical level may include:

$$“ [ \#6 X 3 x 0 : 2 ] ( - [ \#6 X 4 x 0 : 1 ] ) - [ \#7 X 3 x 2 : 3 ] ” .$$

The above encoding of different hierarchical levels is from the dimension of structural fragment expansion and the dimension from the chemical element type to the structural type representation of the atoms. In practical scenarios, the encoding of different hierarchical levels may include the expansion of the above two dimensions at the same time. For example, compared with the second hierarchical level, the third hierarchical level may include structural fragment expansion and structural type representation expansion at the same time.

Through the above encoding method, the structural information of the target molecule may be described systematically and accurately, and detailed and reliable basic data may be provided for subsequent force field parameter matching and molecular simulation. By using this encoding method, the encoded representation of the molecule may be refined layer by layer without increasing the computational complexity, which makes the matching process of the force field parameter more efficient and accurate, significantly improving the accuracy and efficiency of the molecular simulation.

In an embodiment, the electronic device 110 encoding the structural information includes: for a given hierarchical level in the plurality of hierarchical levels, encoding feature information of each atom indicated by the given hierarchical level to determine encoded representations corresponding to each atom; and in response to there are at least two atoms having the same encoded representation, supplementing feature information of the at least two atoms such that the encoded representations corresponding to each atom indicated by the given hierarchical level are different from each other.

Taking the determination of the encoded representation of a given hierarchical level in the plurality of hierarchical levels as an example, the determination process of the encoded representation of any hierarchical level will be described. Firstly, feature information of an atom to be encoded indicated by the given hierarchical level is acquired. The atom to be encoded corresponds to the atom indicated by the given hierarchical level, for example, the first structural fragment 301 corresponding to the top hierarchical level in FIG. 4 includes one carbon atom, one nitrogen atom and another carbon atom connected to the carbon atom. The feature information may include a chemical element type representation, a bonding condition, aromaticity, a number of ring connections, etc. of the atom.

For each atom indicated by the given hierarchical level, the feature information of each atom is acquired. Encoding is performed on the feature information of each atom to obtain the encoded representations corresponding to each atom. The encoded representation should reflect the chemical features and structural features of the atom. The encoded representation of each atom in the given hierarchical level is checked to determine whether there are at least two atoms with the same encoded representation. If there are the same encoded representations, further processing is required to ensure the uniqueness of the encoded representation.

For the atoms with the same encoded representation, the feature supplement may be performed such that the encoded representations are different from each other. The content of the supplement may include adding more feature information, such as the configuration of the atom in stereochemistry, the type and connection mode of the environmental atom, etc. The supplemented feature information is encoded and added to the original encoded representation to generate an updated and unique encoded representation.

In this way, it can be ensured that the encoded representations of all atoms are mutually exclusive at each hierarchical level, thereby avoiding incorrect matching caused by repeated encoded representations. The method improves the accuracy and uniqueness of encoding, which makes the matching process of the molecular force field parameter more efficient and accurate.

After determining the encoded representations of the plurality of hierarchical levels, the electronic device 110 may further traverse the plurality of hierarchical levels to obtain the encoded representations corresponding to each hierarchical level in the plurality of hierarchical levels. In response to there are at least two hierarchical levels having partially identical encoded representations in the encoded representations corresponding to each hierarchical level, and a difference indicating the structural type representation of the atom, the encoded representations corresponding to the at least two hierarchical levels are associated, where the structural type representation is a part of the feature information, and the association includes determining the encoded representation corresponding to the first hierarchical level as a root node, and determining the encoded representation corresponding to other hierarchical levels as child nodes corresponding to the root node, where the first hierarchical level is the top hierarchical level of other hierarchical levels.

After determining the encoded representations of the plurality of hierarchical levels, the hierarchical levels may be traversed downwards from the top hierarchical level. Encoding is performed on the atoms indicated by each hierarchical level to obtain the encoded representation of the hierarchical level. In the traversing process, the encoded representation of each hierarchical level is checked to determine whether there are at least two hierarchical levels with the same encoded representation. If there are the same encoded representations, they may also be marked, and the difference may be determined.

For example, the electronic device 110 finds that the encoded representations of the second hierarchical level and the third hierarchical level are partially the same during the traversing process, but the difference indicates the structural type of the atom. That is, the difference indicates that the third hierarchical level adds a feature corresponding to the indication of the bond, the bond angle or the dihedral angle. In this case, an association may be performed on the second hierarchical level and the third hierarchical level to merge these hierarchical levels into the same node of the tree structure. Specifically, the encoded representation of the second hierarchical level is determined as the root node, and the encoded representation of the third hierarchical level is determined as the child node corresponding to the root node.

Through the above process, if there are partially identical encoded representations between different hierarchical levels, and the different encoded representations are caused by the structural type representation, the encoded representations of different hierarchical levels may be associated, thereby constructing a tree structure of the encoded representations between different hierarchical levels. Through the tree structure, an association relationship is established for the original independent encoded representations. The same part of the encoded representation is shared between different hierarchical levels, which avoids repeated matching in the subsequent molecular force field parameter matching process, thereby improving the matching efficiency.

In an embodiment, the process of determining the molecular force field parameter may include: acquiring a molecular force field parameter matching table, where the molecular force field parameter matching table indicates a matching relationship between the molecular force field parameter and the encoded representation; and determining, from the molecular force field parameter matching table, the molecular force field parameter matching the encoded representation of the target molecule as the molecular force field parameter corresponding to the target molecule.

The molecular force field parameter matching table indicates the matching relationship between the molecular force field parameter and the encoded representations. Specifically, the molecular force field parameter matching table records the molecular force field parameters corresponding to each encoded representation in the molecular force field parameter matching table, which is convenient for quick search and matching. Based on the determined encoded representation of the target molecule, the molecular force field parameter matching the encoded representation may be quickly determined according to the molecular force field parameter matching table.

In an embodiment, the process of determining the molecular force field parameter based on the molecular force field parameter matching table may specifically include: from the top hierarchical level in the plurality of hierarchical levels, sequentially performing force field parameter matching in the force field parameter matching table based on the encoded representations corresponding to each hierarchical level until a matching stop condition is satisfied, where the matching stop condition indicates that there is no molecular force field parameter in the force field parameter matching table matching the encoded representation corresponding to the hierarchical level where matching is performed; and determining, based on a matching result of an immediately preceding hierarchical level of the hierarchical level where matching is performed, the molecular force field parameter corresponding to the target molecule in the force field parameter matching table.

For the determined encoded representation corresponding to the plurality of hierarchical levels, the matching may be performed in the force field parameter matching table firstly from the encoded representation of the top hierarchical level. If the encoded representation of the top hierarchical level finds the corresponding molecular force field parameter in the force field parameter matching table, the matching is continued on the encoded representation of a next hierarchical level. The encoded representation of each hierarchical level is matched downward in turn, until the matching stop condition is satisfied. The matching stop condition indicates that when there is no molecular force field parameter in the force field parameter matching table matching the encoded representation corresponding to the hierarchical level where matching is performed, the matching process stops. For example, if the matching molecular force field parameter cannot be found at a certain hierarchical level, the matching process is stopped. When the matching stop condition is satisfied, the molecular force field parameter corresponding to the target molecule is determined based on the matching result of the immediately preceding hierarchical level. That is, if the matching molecular force field parameter cannot be found at a certain hierarchical level, the matching result of a preceding hierarchical level of the hierarchical level is used as the molecular force field parameter of the target molecule.

Through the hierarchical matching, the structural feature of the target molecule can be captured with greater granularity, thereby improving the matching accuracy of molecular force field parameters. In addition, the hierarchical matching avoids the computational complexity caused by matching the entire molecular structure at one time, and reduces the computational burden. The setting of the matching stop condition can ensure that when a better matching result cannot be found, the matching result of a higher hierarchical level is used, which ensures the reliability of the matching result.

The process of the hierarchical matching may be performed based on a machine learning model. The training process of the machine learning model will be briefly introduced below. Firstly, the molecular samples in the model training dataset are preprocessed to extract the structural information thereof. Thereafter, encoding is performed on the structural information of each molecular sample to obtain the encoded representation of the molecular sample. For a given molecular sample of each molecular sample, the encoded representation of the given molecular sample includes encoded representations corresponding to a plurality of hierarchical levels, a designated hierarchical level in the plurality of hierarchical levels indicates feature information of at least one atom included in the given molecular sample, and the feature information indicated by the designated hierarchical level includes additional feature information based on feature information indicated by an immediately preceding hierarchical level. That is, for each molecular sample, the encoded representation of the molecular sample includes the encoded representations corresponding to the plurality of hierarchical levels.

Taking 3 hierarchical levels as an example, for the top hierarchical level, the following training process is performed: firstly, initial molecular force field parameters are allocated to each encoded representation of the top hierarchical level; and the parameters in the machine learning model are adjusted based on a difference between the initial molecular force field parameters and a labeling result, until a difference between the molecular force field parameters determined by the machine learning model based on the encoded representation and the labeling result converges.

For the second hierarchical level, the following training process is performed: the molecular force field parameters corresponding to each encoded representation of the second hierarchical level are determined based on the training result of the top hierarchical level; and the parameters in the machine learning model are adjusted again based on a difference between the determined molecular force field parameters and the labeling result, until a difference between the molecular force field parameters determined by the machine learning model based on the encoded representation and the labeling result converges. Similarly, the molecular force field parameters corresponding to each encoded representation of the third hierarchical level is determined based on the training result of the second hierarchical level, and the above parameter adjustment process is repeated until the difference between the molecular force field parameters determined by the machine learning model and the labeling result converges.

Through the above hierarchical training method, it can be ensured that the parameters of the corresponding machine learning model are optimized in the training process, and when the early hierarchical level is trained, a good initial value is provided for the training of the subsequent hierarchical level. The tree structure training method has higher efficiency and better precision compared with the traditional linear training method, because the parameters in the machine learning model are fully optimized, which can better adapt to the diversity of the molecular structure.

In the process of matching the molecular force field parameter of the target molecule, the force field parameter corresponding to the top hierarchical level may be determined (the force field parameters corresponding to the top hierarchical level are determined based on the encoding table corresponding to the top hierarchical level) from the top hierarchical level in the encoded representation of the target molecule based on the trained machine learning model. Thus, the force field parameter matching is performed in the force field parameter matching table based on the determined force field parameter. And so on, until the matching stop condition is satisfied.

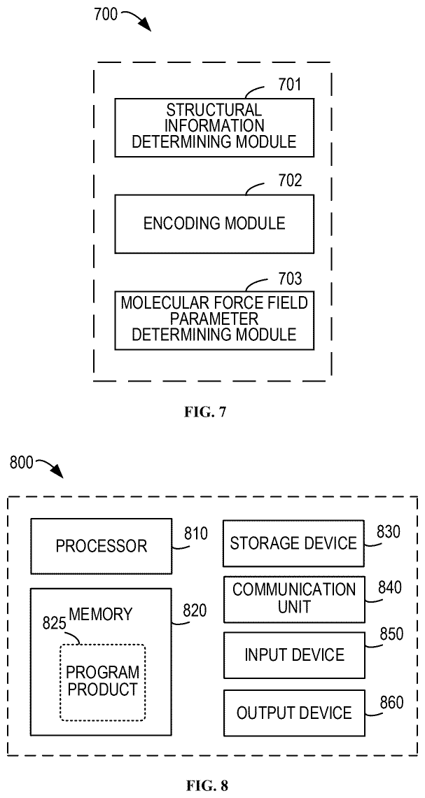

FIG. 7 shows a schematic structural block diagram of a molecular encoding apparatus 700 according to some embodiments in the disclosure. The apparatus 700 may be implemented or included in the electronic device 110, for example. Each module/component in the apparatus 700 may be implemented by hardware, software, firmware, or any combination thereof.

As shown in the figure, the apparatus 700 includes a structural information determining module 701 configured to acquire structural information of a target molecule; an encoding module 702 configured to encode the structural information to obtain an encoded representation of the target molecule, where the encoded representation includes encoded representations corresponding to a plurality of hierarchical levels, a designated hierarchical level in the plurality of hierarchical levels indicates feature information of at least one atom included in the target molecule, and the feature information indicated by the designated hierarchical level includes additional feature information based on feature information indicated by an immediately preceding hierarchical level; and a molecular force field parameter determining module 703 configured to determine, based on the encoded representation of the target molecule, a molecular force field parameter corresponding to the target molecule.

In some embodiments in the disclosure, the encoding module 702 is further configured to: for a top hierarchical level of the plurality of hierarchical levels, acquire a first structural fragment in the target molecule, where the first structural fragment includes at least one atom; and encode feature information of the at least one atom included in the first structural fragment to obtain an encoded representation corresponding to the top hierarchical level. In addition, for a non-top hierarchical level of the plurality of hierarchical levels, acquire a second structural fragment in the target molecule, where the second structural fragment is determined based on a structural fragment corresponding to an immediately preceding hierarchical level of the non-top hierarchical level; and encode feature information of an atom included in the second structural fragment to obtain an encoded representation corresponding to the non-top hierarchical level.

In some embodiments in the disclosure, the encoding module 702 is further configured to: for a top hierarchical level of the plurality of hierarchical levels, acquire a chemical element type representation of a given atom in at least one atom indicated by the top hierarchical level, where the chemical element type representation is a part of the feature information; and encode the chemical element type representation of the given atom to obtain the encoded representation corresponding to the top hierarchical level. In addition, for a non-top hierarchical level of the plurality of hierarchical levels, acquire a structural type representation of a given atom in at least one atom indicated by the top hierarchical level, where the structural type representation is a part of the feature information; and encode the structural type representation and the chemical element type representation of the given atom to obtain the encoded representation corresponding to the non-top hierarchical level.

In some embodiments in the disclosure, the encoding module 702 is further configured to: for a given hierarchical level in the plurality of hierarchical levels, encode feature information of each atom indicated by the given hierarchical level, to determine encoded representations corresponding to each atom; and in response to there are at least two atoms having the same encoded representation, supplement feature information of the at least two atoms such that the encoded representations corresponding to each atom indicated by the given hierarchical level are different from each other.

In some embodiments in the disclosure, the apparatus 700 further includes an encoded representation association module configured to traverse the plurality of hierarchical levels to obtain the encoded representations corresponding to each hierarchical level in the plurality of hierarchical levels; and in response to there are at least two hierarchical levels having partially identical encoded representations in the encoded representations corresponding to each hierarchical level, and a difference indicating the structural type representation of the atom, associate the encoded representations corresponding to the at least two hierarchical levels, where the structural type representation is a part of the feature information, and the association includes determining the encoded representation corresponding to the first hierarchical level as a root node, and determining the encoded representation corresponding to other hierarchical levels as child nodes corresponding to the root node, where the first hierarchical level is the top hierarchical level of other hierarchical levels.

In some embodiments in the disclosure, the molecular force field parameter determining module 703 may include a matching table acquiring sub-module configured to acquire a molecular force field parameter matching table, where the molecular force field parameter matching table indicates a matching relationship between the molecular force field parameter and the encoded representation; and a matching executing sub-module configured to determine, from the molecular force field parameter matching table, the molecular force field parameter matching the encoded representation of the target molecule as the molecular force field parameter corresponding to the target molecule.

In some embodiments in the disclosure, the matching executing sub-module is further configured to: from the top hierarchical level in the plurality of hierarchical levels, sequentially perform force field parameter matching in the force field parameter matching table based on the encoded representations corresponding to each hierarchical level until a matching stop condition is satisfied, where the matching stop condition indicates that there is no molecular force field parameter in the force field parameter matching table matching the encoded representation corresponding to the hierarchical level where matching is performed; and determine, based on a matching result of an immediately preceding hierarchical level of the hierarchical level where matching is performed, the molecular force field parameter corresponding to the target molecule in the force field parameter matching table.

FIG. 8 shows a block diagram of an electronic device 800 in which one or more embodiments in the disclosure may be implemented. It should be understood that the electronic device 800 shown in FIG. 8 is merely an example, and should not constitute any limitation on the function and scope of the embodiments described herein. The electronic device 800 shown in FIG. 8 may include or be implemented as the electronic device 110 of FIG. 1 or the apparatus 700 of FIG. 7.

As shown in FIG. 8, the electronic device 800 is in the form of a general-purpose electronic device. The components of the electronic device 800 may include, but are not limited to, one or more processors or processing units 810, a memory 820, a storage device 830, one or more communication units 840, one or more input devices 850, and one or more output devices 860. The processor 810 may be an actual or virtual processor and may execute various processes according to programs stored in the memory 820. In a multi-processor system, multiple processors execute computer-executable instructions in parallel to improve the parallel processing capability of the electronic device 800.

The electronic device 800 generally includes a plurality of computer storage media. Such media may be any available media accessible by the electronic device 800, including but not limited to volatile and non-volatile media, removable and non-removable media. The memory 820 may be volatile memory (for example, register, cache, random access memory (RAM)), non-volatile memory (for example, read-only memory (ROM), electrically erasable programmable read-only memory (EEPROM), flash memory), or a combination thereof. The storage device 830 may be removable or non-removable media, and may include a machine-readable medium, such as a flash drive, a magnetic disk, or any other medium, which may be capable of storing information and/or data and may be accessed within the electronic device 800.

The electronic device 800 may further include additional removable/non-removable, volatile/non-volatile storage media. Although not shown in FIG. 8, a magnetic disk drive for reading or writing from a removable, non-volatile magnetic disk (e.g., a “floppy disk”) and an optical disk drive for reading or writing from a removable, non-volatile optical disk may be provided. In these cases, each drive may be connected to a bus (not shown) by one or more data medium interfaces. The memory 820 may include a computer program product 825 having one or more program modules configured to perform various methods or actions of various embodiments in the disclosure.

The communication unit 840 enables communication with other electronic devices through a communication medium. Additionally, the functionality of the components of the electronic device 800 may be implemented in a single computing cluster or multiple computing machines that are capable of communicating through a communication connection. Thus, the electronic device 800 may operate in a networked environment using logical connections to one or more other servers, network personal computers (PCs), or another network node.

The input device 850 may be one or more input devices, such as a mouse, a keyboard, a trackball, or the like. The output device 860 may be one or more output devices, such as a display, a speaker, a printer, or the like. The electronic device 800 may also communicate with one or more external devices (not shown), such as storage devices, display devices, etc., communicate with one or more devices that enable a user to interact with the electronic device 800, or communicate with any device (e.g., network card, modem, etc.) that enables the electronic device 800 to communicate with one or more other electronic devices, as required, through the communication unit 840. Such communication may be performed via an input/output (I/O) interface (not shown).

According to an example embodiment in the disclosure, a computer-readable storage medium is provided, on which computer-executable instructions are stored, where the computer-executable instructions are executed by a processor to implement the methods described above. According to an example embodiment in the disclosure, a computer program product is further provided, which is tangibly stored on a non-transitory computer-readable medium and includes computer-executable instructions, and the computer-executable instructions are executed by a processor to implement the methods described above.

According to an example embodiment in the disclosure, a computer program product or a computer program is provided, the computer program product or the computer program including computer instructions, the computer instructions being stored in a computer-readable storage medium. A processor of a computer device reads the computer instructions from the computer-readable storage medium, and executes the computer instructions to cause the computer device to perform the methods provided in various optional manners in FIG. 2, which will not be described here.

Various aspects in the disclosure are described herein with reference to the flowchart and/or block diagram of the method, apparatus, device, and computer program product implemented according to the disclosure. It should be understood that each block of the flowchart and/or block diagram and the combination of blocks in the flowchart and/or block diagram may be implemented by computer-readable program instructions.

These computer-readable program instructions may be provided to the processor of a general-purpose computer, a special-purpose computer, or other programmable data processing apparatus, thereby producing a machine, such that the instructions, when executed by the processor of the computer or other programmable data processing apparatus, produce an apparatus for implementing the functions/actions specified in one or more blocks of the flowchart and/or block diagram. These computer-readable program instructions may also be stored in a computer-readable storage medium, and these instructions cause the computer, the programmable data processing apparatus, and/or other devices to work in a specific manner, and thus, the computer-readable medium storing the instructions includes an article of manufacture, which includes instructions for implementing various aspects of the functions/actions specified in one or more blocks of the flowchart and/or block diagram.

The computer-readable program instructions may be loaded onto the computer, other programmable data processing apparatus, or other device, such that a series of operation steps are performed on the computer, other programmable data processing apparatus or other device to produce a computer-implemented process, such that the instructions executed on the computer, other programmable data processing apparatus, or other device implement the functions/actions specified in one or more blocks of the flowchart and/or block diagram.

The flowchart and block diagrams in the drawings show the architecture, functionality, and operation of possible implementations of systems, methods, and computer program products according to multiple embodiments in the disclosure. In this regard, each block in the flowchart or block diagram may represent a module, program segment, or portion of instructions, and the module, program segment, or portion of instructions includes one or more executable instructions for implementing the specified logical function. In some alternative embodiments, the functions noted in the blocks may also occur out of the order noted in the figures. For example, two blocks shown in succession may, in fact, be executed substantially concurrently, or the blocks may sometimes be executed in a reverse order, depending upon the functionality involved. It is also noted that, each block of the block diagrams and/or flowcharts, and combinations of blocks in the block diagrams and/or flowcharts, may be implemented by special purpose hardware-based systems that perform the specified functions or acts, or combinations of special purpose hardware and computer instructions.

The various embodiments in the disclosure have been described above, and the above description is an example, not exhaustive, and is not limited to the disclosed embodiments. Many modifications and variations will be apparent to those of ordinary skill in the art without departing from the scope and spirit of the described embodiments. The terminology used herein was chosen to best explain the principles of the embodiments, the practical application, or improvements made to the technology in the market, or to enable others of ordinary skill in the art to understand the embodiments disclosed herein.

1. A method for determining a molecular force field parameter, comprising:
acquiring structural information of a target molecule;
encoding the structural information to obtain an encoded representation of the target molecule, wherein the encoded representation comprises encoded representations corresponding to a plurality of hierarchical levels, a designated hierarchical level in the plurality of hierarchical levels indicates feature information of at least one atom comprised in the target molecule, and the feature information indicated by the designated hierarchical level comprises additional feature information based on feature information indicated by an immediately preceding hierarchical level; and
determining, based on the encoded representation of the target molecule, the molecular force field parameter corresponding to the target molecule.

2. The method of claim 1, wherein encoding the structural information comprises:
for a top hierarchical level in the plurality of hierarchical levels,
acquiring a first structural fragment in the target molecule, wherein the first structural fragment comprises at least one atom;
encoding the feature information of the at least one atom comprised in the first structural fragment to obtain an encoded representation corresponding to the top hierarchical level;
for a non-top hierarchical level in the plurality of hierarchical levels,
acquiring a second structural fragment in the target molecule, wherein the second structural fragment is determined based on a structural fragment corresponding to an immediately preceding hierarchical level of the non-top hierarchical level; and
encoding feature information of an atom comprised in the second structural fragment to obtain an encoded representation corresponding to the non-top hierarchical level.

3. The method of claim 1, wherein encoding the structural information comprises:
for a top hierarchical level in the plurality of hierarchical levels,
acquiring a chemical element type representation of a given atom in at least one atom indicated by the top hierarchical level, wherein the chemical element type representation is a part of the feature information;
encoding the chemical element type representation of the given atom to obtain an encoded representation corresponding to the top hierarchical level;
for a non-top hierarchical level in the plurality of hierarchical levels,
acquiring a structural type representation of a given atom in at least one atom indicated by the top hierarchical level, wherein the structural type representation is a part of the feature information; and
encoding the structural type representation and the chemical element type representation of the given atom to obtain an encoded representation corresponding to the non-top hierarchical level.

4. The method of claim 1, wherein encoding the structural information comprises:
for a given hierarchical level in the plurality of hierarchical levels,
encoding feature information of each atom indicated by the given hierarchical level to determine encoded representations corresponding to each atom; and
in response to there are at least two atoms having the same encoded representation, supplementing feature information of the at least two atoms such that the encoded representations corresponding to each atom indicated by the given hierarchical level are different from each other.

5. The method of claim 1, further comprising:
traversing the plurality of hierarchical levels to obtain encoded representations corresponding to each hierarchical level in the plurality of hierarchical levels; and
in response to there are at least two hierarchical levels having partially identical encoded representations in the encoded representations corresponding to each hierarchical level and a difference indicating a structural type representation of an atom, associating the encoded representations corresponding to the at least two hierarchical levels, wherein the structural type representation is a part of the feature information, and the association comprises determining an encoded representation corresponding to a first hierarchical level as a root node, and determining an encoded representation corresponding to other hierarchical levels as child nodes corresponding to the root node, wherein the first hierarchical level is a top hierarchical level of the other hierarchical levels.

6. The method of claim 1, wherein determining the molecular force field parameter corresponding to the target molecule comprises:
acquiring a molecular force field parameter matching table, wherein the molecular force field parameter matching table indicates a matching relationship between a molecular force field parameter and an encoded representation; and
determining, from the molecular force field parameter matching table, a molecular force field parameter matching the encoded representation of the target molecule as the molecular force field parameter corresponding to the target molecule.

7. The method of claim 6, wherein determining, from the molecular force field parameter matching table, the molecular force field parameter matching the encoded representation of the target molecule comprises:
from a top hierarchical level in the plurality of hierarchical levels, sequentially performing force field parameter matching in the force field parameter matching table based on the encoded representations corresponding to each hierarchical level until a matching stop condition is satisfied, wherein the matching stop condition indicates that there is no molecular force field parameter in the force field parameter matching table matching the encoded representation corresponding to the hierarchical level where matching is performed; and
determining, based on a matching result of an immediately preceding hierarchical level of the hierarchical level where matching is performed, the molecular force field parameter corresponding to the target molecule in the force field parameter matching table.

8. An electronic device, comprising:
at least one processor; and
at least one memory coupled to the at least one processor and storing instructions for execution by the at least one processor, the instructions, when executed by the at least one processor, causing the electronic device to perform operations comprising:
acquiring structural information of a target molecule;
encoding the structural information to obtain an encoded representation of the target molecule, wherein the encoded representation comprises encoded representations corresponding to a plurality of hierarchical levels, a designated hierarchical level in the plurality of hierarchical levels indicates feature information of at least one atom comprised in the target molecule, and the feature information indicated by the designated hierarchical level comprises additional feature information based on feature information indicated by an immediately preceding hierarchical level; and
determining, based on the encoded representation of the target molecule, the molecular force field parameter corresponding to the target molecule.

9. The electronic device of claim 8, wherein encoding the structural information comprises:
for a top hierarchical level in the plurality of hierarchical levels,
acquiring a first structural fragment in the target molecule, wherein the first structural fragment comprises at least one atom;
encoding the feature information of the at least one atom comprised in the first structural fragment to obtain an encoded representation corresponding to the top hierarchical level;
for a non-top hierarchical level in the plurality of hierarchical levels,
acquiring a second structural fragment in the target molecule, wherein the second structural fragment is determined based on a structural fragment corresponding to an immediately preceding hierarchical level of the non-top hierarchical level; and
encoding feature information of an atom comprised in the second structural fragment to obtain an encoded representation corresponding to the non-top hierarchical level.

10. The electronic device of claim 8, wherein encoding the structural information comprises:
for a top hierarchical level in the plurality of hierarchical levels,
acquiring a chemical element type representation of a given atom in at least one atom indicated by the top hierarchical level, wherein the chemical element type representation is a part of the feature information;
encoding the chemical element type representation of the given atom to obtain an encoded representation corresponding to the top hierarchical level;
for a non-top hierarchical level in the plurality of hierarchical levels,
acquiring a structural type representation of a given atom in at least one atom indicated by the top hierarchical level, wherein the structural type representation is a part of the feature information; and
encoding the structural type representation and the chemical element type representation of the given atom to obtain an encoded representation corresponding to the non-top hierarchical level.

11. The electronic device of claim 8, wherein encoding the structural information comprises:
for a given hierarchical level in the plurality of hierarchical levels,
encoding feature information of each atom indicated by the given hierarchical level to determine encoded representations corresponding to each atom; and
in response to there are at least two atoms having the same encoded representation, supplementing feature information of the at least two atoms such that the encoded representations corresponding to each atom indicated by the given hierarchical level are different from each other.

12. The electronic device of claim 8, wherein the operations further comprise:
traversing the plurality of hierarchical levels to obtain encoded representations corresponding to each hierarchical level in the plurality of hierarchical levels; and
in response to there are at least two hierarchical levels having partially identical encoded representations in the encoded representations corresponding to each hierarchical level and a difference indicating a structural type representation of an atom, associating the encoded representations corresponding to the at least two hierarchical levels, wherein the structural type representation is a part of the feature information, and the association comprises determining an encoded representation corresponding to a first hierarchical level as a root node, and determining an encoded representation corresponding to other hierarchical levels as child nodes corresponding to the root node, wherein the first hierarchical level is a top hierarchical level of the other hierarchical levels.

13. The electronic device of claim 8, wherein determining the molecular force field parameter corresponding to the target molecule comprises:
acquiring a molecular force field parameter matching table, wherein the molecular force field parameter matching table indicates a matching relationship between a molecular force field parameter and an encoded representation; and
determining, from the molecular force field parameter matching table, a molecular force field parameter matching the encoded representation of the target molecule as the molecular force field parameter corresponding to the target molecule.

14. The electronic device of claim 13, wherein determining, from the molecular force field parameter matching table, the molecular force field parameter matching the encoded representation of the target molecule comprises:
from a top hierarchical level in the plurality of hierarchical levels, sequentially performing force field parameter matching in the force field parameter matching table based on the encoded representations corresponding to each hierarchical level until a matching stop condition is satisfied, wherein the matching stop condition indicates that there is no molecular force field parameter in the force field parameter matching table matching the encoded representation corresponding to the hierarchical level where matching is performed; and
determining, based on a matching result of an immediately preceding hierarchical level of the hierarchical level where matching is performed, the molecular force field parameter corresponding to the target molecule in the force field parameter matching table.

15. A non-transitory computer-readable storage medium having a computer program stored thereon, wherein the computer program is executable by a processor to perform operations comprising:
acquiring structural information of a target molecule;
encoding the structural information to obtain an encoded representation of the target molecule, wherein the encoded representation comprises encoded representations corresponding to a plurality of hierarchical levels, a designated hierarchical level in the plurality of hierarchical levels indicates feature information of at least one atom comprised in the target molecule, and the feature information indicated by the designated hierarchical level comprises additional feature information based on feature information indicated by an immediately preceding hierarchical level; and
determining, based on the encoded representation of the target molecule, the molecular force field parameter corresponding to the target molecule.

16. The storage medium of claim 15, wherein encoding the structural information comprises:
for a top hierarchical level in the plurality of hierarchical levels,
acquiring a first structural fragment in the target molecule, wherein the first structural fragment comprises at least one atom;
encoding the feature information of the at least one atom comprised in the first structural fragment to obtain an encoded representation corresponding to the top hierarchical level;
for a non-top hierarchical level in the plurality of hierarchical levels,
acquiring a second structural fragment in the target molecule, wherein the second structural fragment is determined based on a structural fragment corresponding to an immediately preceding hierarchical level of the non-top hierarchical level; and
encoding feature information of an atom comprised in the second structural fragment to obtain an encoded representation corresponding to the non-top hierarchical level.

17. The storage medium of claim 15, wherein encoding the structural information comprises:
for a top hierarchical level in the plurality of hierarchical levels,
acquiring a chemical element type representation of a given atom in at least one atom indicated by the top hierarchical level, wherein the chemical element type representation is a part of the feature information;
encoding the chemical element type representation of the given atom to obtain an encoded representation corresponding to the top hierarchical level;
for a non-top hierarchical level in the plurality of hierarchical levels,
acquiring a structural type representation of a given atom in at least one atom indicated by the top hierarchical level, wherein the structural type representation is a part of the feature information; and
encoding the structural type representation and the chemical element type representation of the given atom to obtain an encoded representation corresponding to the non-top hierarchical level.

18. The storage medium of claim 15, wherein encoding the structural information comprises:
for a given hierarchical level in the plurality of hierarchical levels,
encoding feature information of each atom indicated by the given hierarchical level to determine encoded representations corresponding to each atom; and
in response to there are at least two atoms having the same encoded representation, supplementing feature information of the at least two atoms such that the encoded representations corresponding to each atom indicated by the given hierarchical level are different from each other.

19. The storage medium of claim 15, wherein the operations further comprise:
traversing the plurality of hierarchical levels to obtain encoded representations corresponding to each hierarchical level in the plurality of hierarchical levels; and
in response to there are at least two hierarchical levels having partially identical encoded representations in the encoded representations corresponding to each hierarchical level and a difference indicating a structural type representation of an atom, associating the encoded representations corresponding to the at least two hierarchical levels, wherein the structural type representation is a part of the feature information, and the association comprises determining an encoded representation corresponding to a first hierarchical level as a root node, and determining an encoded representation corresponding to other hierarchical levels as child nodes corresponding to the root node, wherein the first hierarchical level is a top hierarchical level of the other hierarchical levels.

20. The storage medium of claim 15, wherein determining the molecular force field parameter corresponding to the target molecule comprises:
acquiring a molecular force field parameter matching table, wherein the molecular force field parameter matching table indicates a matching relationship between a molecular force field parameter and an encoded representation; and
determining, from the molecular force field parameter matching table, a molecular force field parameter matching the encoded representation of the target molecule as the molecular force field parameter corresponding to the target molecule.
